(19) United States
(12) Patent Application Publication
(10) Pub. No.: US 2004/0138145 A1
(43) Pub. Date: Jul. 15, 2004

(54) Application of intestinal biliary acid reuptake inhibitors for the prevention and treatment of alzheimer's disease

(75) Inventors: Thierry Canton, Etrechy (FR); Laurent Pradier, Verrieres (FR); Jesus Benavides, Chatenay Malabry (FR); Hubert Heuer, Schwabenheim (DE); Hans-Ludwig Schaefer, Hochheim (DE)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: Aventis Pharma S.A., Antony (FR)

(21) Appl. No.: 10/734,787

(22) Filed: Dec. 12, 2003

Related U.S. Application Data
(60) Provisional application No. 60/455,354, filed on Mar. 17, 2003.

(30) Foreign Application Priority Data
Dec. 12, 2002 (FR) 0215722

(51) Int. Cl. A61K 31/7052

(52) U.S. Cl. 514/23

(57) ABSTRACT

The subject of the invention is the application of intestinal biliary acid reuptake inhibitors for the prevention and treatment of Alzheimer's disease, where appropriate, in combination with an HMG-CoA reductase inhibitor, a cholesterol uptake inhibitor, a cholesterol synthesis inhibitor or an APP secretase inhibitor.

This application claims the benefit of U.S. Provisional Application No. 60/455,354, filed Mar. 17, 2003 and the benefit of priority of French Patent Application No. 02/15,722, filed Dec. 12, 2002.

The subject of the present invention is the application of intestinal biliary acid reuptake inhibitors for the prevention and treatment of Alzheimer's disease.

Alzheimer's disease (AD) is a progressive neurodegenerative disease which affects a large proportion of the elderly population. This disease is characterized at the clinical level by a loss of memory and a decline in cognitive functions, and at the neuropathological level by the presence in the brain of intracellular neurofibrillary deposits and extracellular deposits of the β-amyloid (A-β) peptide forming the amyloid plaques (Yankner BA (1996) Neuron 16: 921-932). In addition to these signs, there are a large number of other abnormal changes including an impairment of the immune and inflammatory systems and an impairment of the mitochondrial function which can lead to an increase in oxidative stress, an activation of the mechanisms of apoptosis and ultimately to cell death.

Amyloid plaques are predominantly composed of A-β peptides containing 40 or 42 residues which are generated during the proteolytic process for the β-amyloid peptide precursor protein (APP). The extracellular deposits of A-β are very specific for AD and for associated disorders. They represent the invariable feature of all forms of AD, including the familial forms (FAD). The early familial forms of the disease (appearance between 40 and 60 years) are due to mutations in the APP gene and in the presenilin-1 (PS1) and presenilin-2 (PS2) genes. Mutations in these three genes induce changes in the proteolysis of APP, leading to an overproduction of Aβ and to the early appearance of the pathology and symptoms which are similar to those of the sporadic forms of AD (Czech C., et al. (2000) Progress in Neurobiology 60: 361-382).

A link between cholesterol and Alzheimer's disease has also been established from epidemiological studies and from results of recent biochemical and cell biology studies (see review by Hartmann, T. (2001) TINS 24: S45-48). A high cholesterol level at the adult age and a high blood pressure significantly increase the risk of Alzheimer's disease (Kivipelto et al., 2001 Br Med J. 322: 1447).

A greatly reduced risk is recorded in populations under treatment with statin-type hypocholesterolemic agents, however (Wolozin et al. (2000) Arch Neurol. 57: 1439; Jick et al. (2000) Lancet 356: 1627).

The molecular link appears to have been recently established. In vitro and in vivo, a high cholesterol level increases the production of the A-β peptide and accelerates the appearance of amyloid plaques (Sparks et al. (1994) Exp. Neurol. 126: 88-94; Refolo et al. (2000) Neurobiol. Dis. 7: 321-331; Puglielli et al. (2001) Nat. Cell Biol. 3: 905; Shie et al. (2002) Neuroreport 13: 455) while inhibitors of the cholesterol synthesis pathway reduce them (Simons et al. (1998) PNAS USA 95: 6460-6464; Faβbender et al. (2001) PNAS USA 98: 5856, Refolo et al., (2001) Neurobiol. Dis. 8: 890-899).

With the aim of reducing the level of β-amyloid peptide in vivo, and treating, preventing or reducing the progression of Alzheimer's disease, it was therefore suggested to use inhibitors of cholesterol synthesis such as those of 3-hydroxy-3-methylglutaryl coenzyme A reductase (HMG CoA reductase), an enzyme involved in the biosynthesis of cholesterol, as described in WO 00/28981 and in particular statins such as simvastatin (Hartman, 2001 TINS 24: S45-48).

Up until now, it has not been defined if the therapeutic effect of statins was due to a direct action on the central nervous system or if they acted by reducing plasma cholesterol. Indeed, an effect limited to the levels of plasma cholesterol appeared unlikely since it was generally accepted that cerebral cholesterol was independent of plasma cholesterol (Dietschy and Turley (2001) Curr. Opin. Lipidol. 12: 105-112).

### BRIEF DESCRIPTION OF THE DRAWINGS

FIG. 1 illustrates the effect of 0.01% of BARI (compound A) on the plasma cholesterol levels and the soluble Aβ peptide, which is compared with the control regimen group.

FIGS. 2 and 3 respectively show the effects of 0.01% of BARI (compound A) on soluble and total Aβ peptide, which is compared with the control regimen group.

FIG. 4 illustrates the effect of BARI (compound A) at various doses on the levels of total Aβ peptide, which is compared with the control regimen group.

The applicant has shown that a specific pharmacological class, the biliary acid reuptake inhibitors (BARI), which make it possible to reduce the level of plasma cholesterol by blocking the reuptake of biliary acids in the intestine, could also reduce the β-amyloid peptide levels in the brain.

Biliary acid reuptake inhibitors are not absorbed, and their site of action is in the intestine where they block the reuptake of the biliary acids excreted, which constitute a large source of cholesterol precursor.

The results obtained and described below in the experimental part make it possible to demonstrate that the plasma cholesterol levels only have to be reduced in order to reduce the β-amyloid peptide levels in the brain.

Surprisingly, it has therefore been demonstrated that the biliary acid reuptake inhibitors (BARI) are effective in an animal model of Alzheimer's disease by acting only through the regulation of the plasma cholesterol level and in particular by not penetrating into the brain, because they are not absorbed in the body.

The expression, prevention or treatment of Alzheimer's disease is understood to mean the possibility of preventing or delaying the appearance and/or the progression of Alzheimer's disease.

The subject of the invention is therefore the application of compounds which are biliary acid reuptake inhibitors for the preparation of a medicament which makes it possible to prevent or treat Alzheimer's disease.

More generally, the subject of the invention is the application of the compounds or of a mixture of compounds which reduce the plasma cholesterol levels without the need to be absorbed in the body after their oral administration, for preventing or treating Alzheimer's disease.

Molecules having a biliary acid reuptake inhibitory activity (BARI) are in particular described in patents U.S. Pat. No. 6,221,897 and U.S. Pat. No. 6,245,744.

The subject of the invention is therefore more particularly the application of compounds which are biliary acid reuptake inhibitors for the preparation of a medicament which makes it possible to prevent or treat Alzheimer's disease, wherein the biliary acid reuptake inhibitors are compounds of formula (IA):

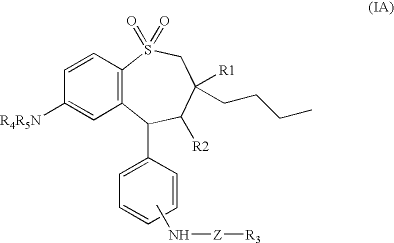

in which:

R¹ represents methyl, ethyl, propyl or butyl;

R² represents H, OH, NH₂, or NH—(C₁-C₆)alkyl;

R³ is a monosaccharide, disaccharides, trisaccharides or quadrisaccharides, said radical being unsubstituted or mono- or polysubstituted with a group for protecting sugars;

R⁴ is methyl, ethyl, propyl or butyl;

R⁵ is methyl, ethyl, propyl or butyl;

Z is (C═O)ₙ—(C₀-C₁₆)-alkyl; (C═O)ₙ—(C₀-C₁₆)-alkyl-NH; (C═O)ₙ—(C₀-C₁₆)-alkyl-O; (C═O)ₙ—(C₀-C₁₆)-alkyl-(C═O)ₘ—; or a covalent bond;

n is 0 or 1;

m is 0 or 1;

and their pharmaceutically acceptable addition salts.

The expression monosaccharide radical is understood to mean polyalcohols containing 5, 6, 7 or 8 carbon atoms, also comprising carbonyl (ketone or aldehyde) groups, which most often do not exist in the free state but are combined with one or more hydroxyl groups of the same molecule, in the form of a hemiketal or a cyclic hemiketal. This may include sugars containing 5 carbon atoms such as L-arabinose, D-ribose, 2-deoxy-D-ribose and D-xylose.

These sugars form part of the pentose (or aldopentose) series.

It may also include sugars containing 6 carbons, such as D-glucose, D-fructose, D-galactose and D-mannose. It may also include erythrose, glyceraldehyde, sedoheptulose, glucosamine, galactosamine, glucoronic acid, galacturonic acid, gluconic acid, galactonic acid, mannonic acid, glucamine, 3-amino-1,2-propanediol, glucaric acid and galactaric acid. Among the preferred carbohydrates the following radicals may be mentioned:

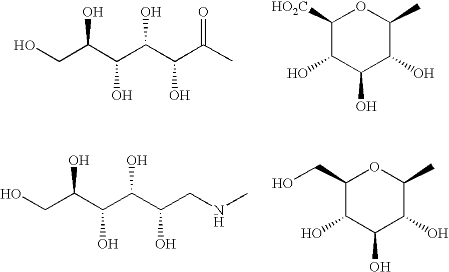

The subject of the invention is most particularly the application of a compound which is a biliary acid reuptake inhibitor for the preparation of a medicament which makes it possible to prevent or treat Alzheimer's disease, wherein the biliary acid reuptake inhibitor is the following compound of formula (IA), compound A:

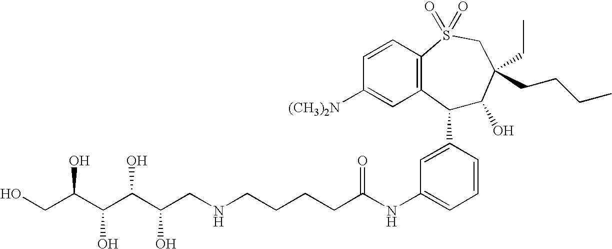

The subject of the invention is also more particularly the application of compounds which are biliary acid reuptake inhibitors for the preparation of a medicament which makes it possible to prevent or treat Alzheimer's disease, wherein the biliary acid reuptake inhibitors are compounds of formula (IB):

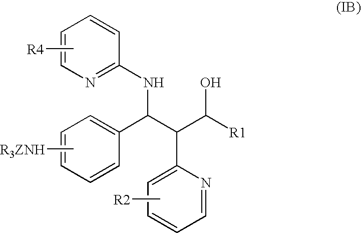

in which R¹ is a phenyl radical or a heteroaryl group which is unsubstituted or substituted with one to three independent radicals chosen from F, Cl, Br, I, —OH, —CF₃, —NO₂, —NHR⁹, —NR⁹R¹⁰, —CHO, —CO₂H, —CO₂R¹¹, —COR¹², —(C₁-C₆)-alkyl-OH, —(C₁-C₆)-alkyl-OH-phenyl, —(C₁-C₆)-alkyl-CF₃, —(C₁-C₆)-alkyl-NO₂, —(C₁-C₆)-alkyl-CN, —(C₁-C₆)-alkyl-NH₂, —(C₁-C₆)-alkyl-NHR⁹, —(C₁-C₆)-alkyl-NR⁹R¹⁰, —(C₁-C₆)-alkyl-CHO, —(C₁-C₆)-alkyl-CO₂H, —(C₁-C₆)-alkyl-CO₂R¹¹, —(C₁-C₆)-alkyl-COR¹², —O—(C₁-C₆)-alkyl-OH, —O—(C₁-C₆)-alkyl(—OH)-phenyl, —O—(C₁-C₆)-alkyl-CF₃, —O—(C₁-C₆)-alkyl-NO₂, —O—(C₁-C₆)-alkyl-CN, —O—(C₁-C₆)-alkyl-NH₂, —O—(C₁-C₆)-alkyl-NHR⁹, —O—(C₁-C₆)-alkyl-NR⁹R¹⁰, —(C₁-C₆)-alkyl-CHO, —O—(C₁-C₆)—N—S₃H, —S₂—CH₃, —O—(C₁-C₆)-alkyl-O—(C₁-C₆)-alkylphenyl, —(C₁-C₆)-alkylthio or pyridyl, it being possible for said alkyl derivatives to be substituted with one or more fluorine atoms and it being possible for the phenyl or pyridyl groups to be monosubstituted with methyl, methoxy or halogen;

R² represents H, OH, —CH₂OH, —OMe, —CHO or —NH₂;

R³ is a monosaccharide residue, disaccharides, trisaccharides or quadrisaccharides, said radical being unsubstituted or mono- or polysubstituted with a group for protecting sugars, HO—SO₂— or (HO)₂—PO—;

R⁴ is H, methyl, F or —OMe;

R⁹ to R¹² represent, independently of each other, H or —(C₁-C₈)-alkyl;

Z represents a covalent bond or a group —NH—(C₀-C₁₆)-alkyl-CO—, —O—(C₀-C₁₆)-alkyl-CO—, —(CO)ₘ—(C₀-C₁₆)-alkyl-(CO)ₙ—, an amino acid residue, a diamino acid residue, it being understood that said amino acid residue or diamino acid residue may be mono- or polysubstituted with an amino acid-protecting group, n is 0 or 1, m is 0 or 1, and their pharmaceutically acceptable addition salts.

The subject of the invention is more particularly the application of a compound which is a biliary acid reuptake inhibitor for the preparation of a medicament which makes it possible to prevent or treat Alzheimer's disease, wherein the biliary acid reuptake inhibitor is the following compound of formula (IB), compound B:

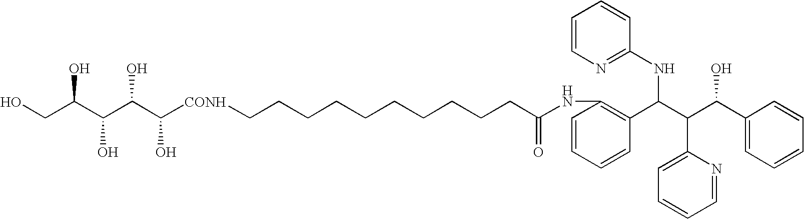

The preparations of these compounds are described in the patents cited above.

The biliary acid reuptake inhibitors in their application according to the invention may be administered neat or in combination with one or more other compounds chosen from:

HMG-CoA reductase inhibitors such as the statins,

cholesterol uptake inhibitors,

inhibitors of the synthesis of cholesterol and any other agent reducing the plasma and/or cerebral cholesterol levels,

γ and β APP secretase inhibitors.

Ezetimibe may be mentioned among the cholesterol uptake inhibitors. Among the γ and β APP secretase inhibitors, there may be mentioned the compounds as described by H. Josien (2002, Current Opinion in Drug Disc. & dev 5: 513-525) or in the general review by M. S. Wolfe, (2002, Nat. Rev. Drug. Discov. 1: 859-866).

The subject of the invention is therefore also the application of compounds which are biliary acid reuptake inhibitors for the preparation of a medicament which makes it possible to prevent or treat Alzheimer's disease, wherein the biliary acid reuptake inhibitors are combined with one or more other compounds chosen from

a) HMG-CoA reductase inhibitors, or

b) cholesterol uptake inhibitors, or

c) cholesterol synthesis inhibitors, or

d) APP secretase inhibitors.

The subject of the invention is therefore also the application of compounds which are biliary acid reuptake inhibitors for the preparation of a medicament which makes it possible to prevent or treat Alzheimer's disease, wherein the biliary acid reuptake inhibitors are combined with an HMG-CoA reductase inhibitor, a cholesterol uptake inhibitor, a cholesterol synthesis inhibitor or a γ and β APP secretase inhibitor for administration simultaneously, separately or spaced out over time.

The subject of the invention is also a method for the prevention or treatment of Alzheimer's disease for a patient at risk of developing this disease or in the course of developing the disease, comprising the administration, to this patient, of an effective therapeutic quantity of a compound having a hypocholesterolemic activity and not penetrating into the body after their oral administration.

More precisely, the subject of the invention is a method for the prevention or treatment of Alzheimer's disease as defined above, wherein the compound having a hypocholesterolemic activity and not penetrating into the body is a biliary acid reuptake inhibitor.

Most particularly, the subject of the invention is a method for the prevention or treatment of Alzheimer's disease for a patient at risk of developing this disease or in the course of developing this disease, comprising the administration to this patient of a therapeutically effective quantity of a biliary acid reuptake inhibitor as defined in formulae (IA) and (IB) and in particular compound A or compound B.

Moreover, the subject of the invention is a method for the prevention or treatment of Alzheimer's disease as defined above, wherein the biliary acid reuptake inhibitors are administered in combination with one or more compounds chosen from an HMG-CoA reductase inhibitor, a cholesterol uptake inhibitor, a cholesterol synthesis inhibitor or a γ and β APP secretase inhibitor.

The biliary acid reuptake inhibitors may be administered in the form of a pharmaceutical preparation (pharmaceutical composition) which allows administration orally or perorally (for example sublingually).

The subject of the invention is therefore the application of the biliary acid reuptake inhibitors for the preparation of a medicament which makes it possible to prevent or treat Alzheimer's disease, wherein the biliary acid reuptake inhibitors are in the form of pharmaceutical compositions which can be administered orally.

More specifically, the subject of the invention is the application as defined above wherein the pharmaceutical compositions contain an effective dose of at least one biliary acid reuptake inhibitor compound and one or more pharmaceutically inert carriers, and/or one or more customary additives allowing administration orally or perorally.

The pharmaceutical compositions according to the invention normally contain from about 0.01 to about 100 mg, and preferably from about 0.02 to about 50 mg of biliary acid reuptake inhibitor.

The subject of the invention is therefore more particularly the application of the biliary acid reuptake inhibitors for the preparation of a medicament which makes it possible to prevent or treat Alzheimer's disease, wherein the pharmaceutical composition which can be administered orally contains from about 0.02 to about 50 mg of biliary acid reuptake inhibitors.

The pharmaceutical compositions may be administered orally, for example in the form of pills, tablets, coated tablets, film-coated tablets, granules, hard gelatin capsules and soft gelatin capsules., solutions, syrups, an emulsion, a suspension or an aerosol mixture.

The pharmaceutical compositions are prepared according to methods known per se, pharmaceutically inert organic or inorganic carriers being added to the biliary acid reuptake inhibitors.

For the production of pills, tablets, coated tablets and hard gelatin capsules, it is possible to use, for example, lactose, corn starch and its derivatives, talc, stearic acid or its salts, and the like.

The vehicles appropriate for the preparation of solutions, for example emulsions or syrups, are for example water, alcohols, glycerol, polyols, sucrose, invert sugars, glucose, vegetable oils, and the like. The pharmaceutical preparations normally contain from about 0.05 to about 90% by weight of biliary acid reuptake inhibitors.

In addition to the active ingredients and the carriers, the pharmaceutical preparations may contain additives such as, for example, diluents, disintegrants, binders, lubricants, wetting agents, stabilizers, emulsifiers, preservatives, sweetening agents, colorings, flavoring agents, thickeners, buffering agents, and also solvents or solubilizers or agents for obtaining a delayed effect and also salts for modifying the osmotic pressure, coating agents or antioxidants.

The pharmaceutical preparations may also contain two or more biliary acid reuptake inhibitors. Moreover, in addition to at least one or more biliary acid reuptake inhibitors, they may contain at least one or more other active ingredients which can be used therapeutically or prophylactically such as an HMG-CoA reductase inhibitor, a cholesterol uptake inhibitor, a cholesterol synthesis inhibitor or a γ and β APP secretase inhibitor.

When the biliary acid reuptake inhibitors are used, the doses may vary within broad limits and should be set according to the person to be treated. This depends, for example, on the compound used or on the nature and the severity of the disease to be treated and whether severe or chronic conditions exist or whether a prophylactic treatment is used.

In the case of an oral administration, the daily dose varies in general from about 0.1 to about 100 mg/kg, and preferably from about 0.1 to about 50 mg/kg, in particular from about 0.1 to about 5 mg/kg. For example, an adult weighing about 75 kg can envisage a daily dose varying from about 0.3 to about 0.5 mg/kg.

The daily dose may be divided, in particular in the case of the administration of a large quantity of active ingredient, into several, for example 2, 3 or 4 parts. Where appropriate, depending on individual behavior, it may be necessary to administer the different doses in increasing or decreasing amounts.

Tests in vivo of the compound A on the production of the amyloid peptide in a transgenic mouse model were carried out in the following manner:

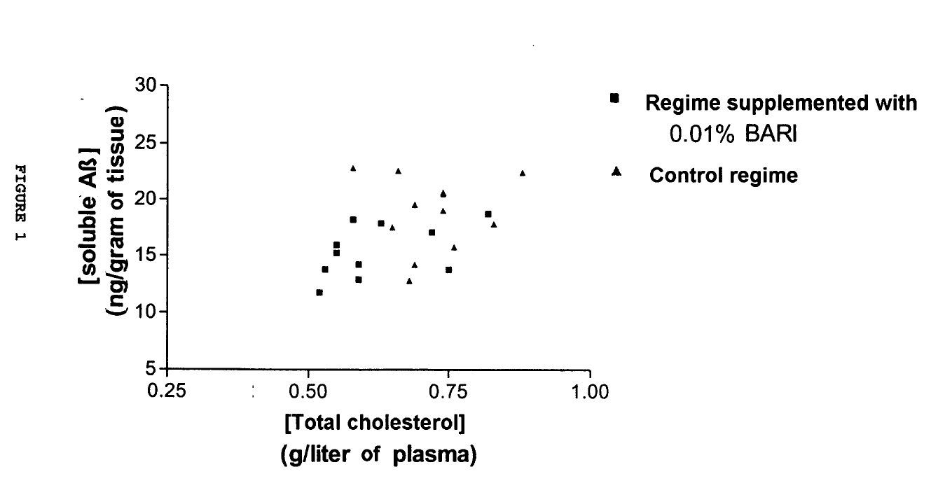

a) Experimental Test 1 (FIG. 1)

Treatment of the Animals

The compound A in powdered form was mixed at the dose of 0.01% (weight/weight) with standard feed in powdered form.

Transgenic mice Tg53 (overexpressing the human APP transgene carrying the “Swedish” and “London” mutations, (2002 Wirths, et al. (2002). Brain Pathol. 12, 275-286), 8-10 week old females, were treated for 3 weeks. The mice were housed in an individual cage with drink being available ad libitum. Every day, 6 grams of powdered food (supplemented or otherwise with compound A) were distributed in each cage. Two groups of 11 to 12 animals (control regimen or regimen supplemented with compound A) were used. At the end of the treatment, a blood sample was collected and the plasma cholesterol level was determined using an automated device for biological analysis.

Preparation of Cerebral Extracts

After being humanely killed, the brain of the mouse was removed and weighed. The tissue was homogenized individually on ice using a Potter device in 10 volumes (weight/volume) of a buffer solution: 0.32 M sucrose, 4 mM Tris-HCl, pH 7.4, containing a cocktail of protease inhibitors (Complete™, Roche Diagnostics). The homogenate was then centrifuged at 50 000×g, for 2 h at 4° C. and the supernatant was collected so as to constitute the soluble (soluble Aβ) brain fraction and was stored at −80° C.

For the measurement of total Aβ, an aliquot of homogenate was denatured with 6M Guanidine Hydrochloride (final concentration), followed by 3 cycles of 15 minutes at 4° C. of ultrasonication

(Bandelin Electronique Sonorex Super RK 102K-Germany) in order to solubilize all the Aβ peptide forms (total fraction).

Assay of the Amyloid Peptide by the ImmunoelectroChemoluminescence Method.

The concentration of the Aβ peptide in the soluble or soluble and insoluble brain fractions from the transgenic mice was determined by immunoelectrochemoluminescence (Yang et al. (1994) Biotechnology (NY) 12 (2), 193-194) using 2 mouse monoclonal antibodies anti-Aβ peptide (4G8 and 6E10) and the reader Origen M8 analyzer (IGEN Europe Inc. Oxford) following a protocol modified according to Khorkova et al. (J. Neurosci. Methods 82, 159-166 (1998) ).

The monoclonal antibody 4G8 (Senetek PLC), which recognizes the epitope residues 17-24 of the Aβ peptide, is ruthenylated by means of the ester TAG-NHS according to the protocol from the supplier (IGEN Europe Inc., Oxford). Ru-4G8 and the biotinylated antibody 6E10, epitope 1-10 of the Aβ peptide (Senetek PLC) are exposed to the soluble brain fraction or the total brain fraction and the tripartite complexes Ru-4G8/Aβ/6E10-biot are quantified by the Origen reader. For the total fraction, the guanidine hydrochloride concentration is brought to 0.3M beforehand by dilution for the assay of the Aβ peptide. A range of synthetic Aβ peptide (Bachem) is used to calibrate each experiment. The Aβ peptide level is calculated in nanogram per g of initial weight of cerebral tissue.

Result

Compared to the control regimen group, the regimen supplemented with compound A (designated as 0.01% BARI in FIG. 1) showed a decrease in the cerebral level of soluble Aβ peptide of 18% [15.45±0.71 ng/g of tissue (n=11) compared with 18.85±0.96 ng/g of tissue (n=12), unpaired t test, p=0.0103].

The plasma cholesterol level was, for its part, also reduced by 14% [regimen supplemented with compound A group: 0.62±0.030 g/l (n=11) compared with the control regimen group: 0.72±0.023 g/l (n=12); unpaired t test p=0.0154] (see FIG. 1)

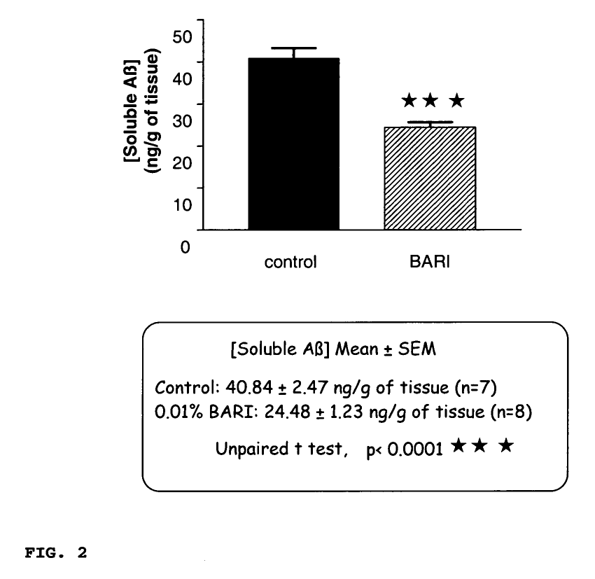

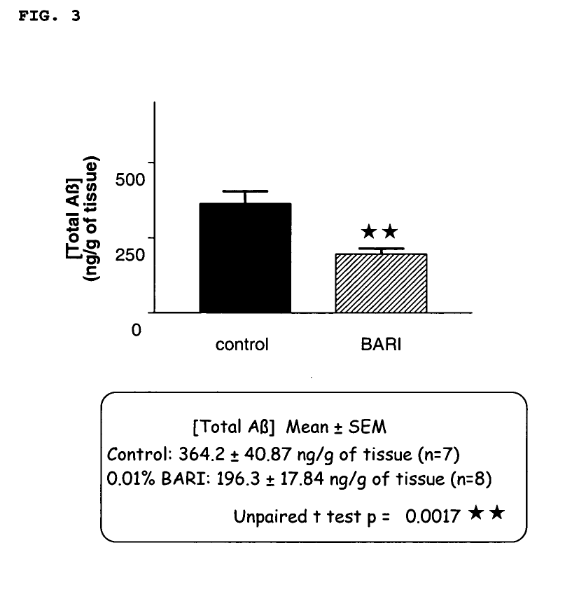

b) Experimental Test No. 2 (FIGS. 2 and 3)

In an experiment using 15.5-week old female transgenic mice at the end of the treatment and therefore with higher Aβ levels due to age, compared with the control regime group, the regime group supplemented with compound A (designated as 0.01%, BARI in FIGS. 2 to 4) showed an even more pronounced reduction in the cerebral level of soluble Aβ peptide, of 40% [24.5±1.2 ng/g of tissue (n=8) compared with 40.8±2.5 ng/g of tissue (n=7), unpaired t test, p=0.0001] (FIG. 2). The cerebral levels of total peptide Aβ (including the soluble forms and the membrane or aggregated forms of the Aβ peptide) are for their part greatly reduced by 46% [196.3±17.8 ng/g of tissue (n=8) compared with 364.2±40.9 ng/g of tissue (n=7), unpaired t test, p=0.0017] (FIG. 3). This effect on the pool of the total forms of Aβ is of importance for the treatment of patients suffering from Alzheimer's disease and who have very high levels of aggregated Aβ peptide in senile plaques.

As above, the plasma cholesterol level was itself reduced by 18% [regime group supplemented with compound A: 0.70±0.03 g/l (n=8) compared with the control regime group: 0.85±0.03 g/l (n=7); unpaired t test, p=0.0037]

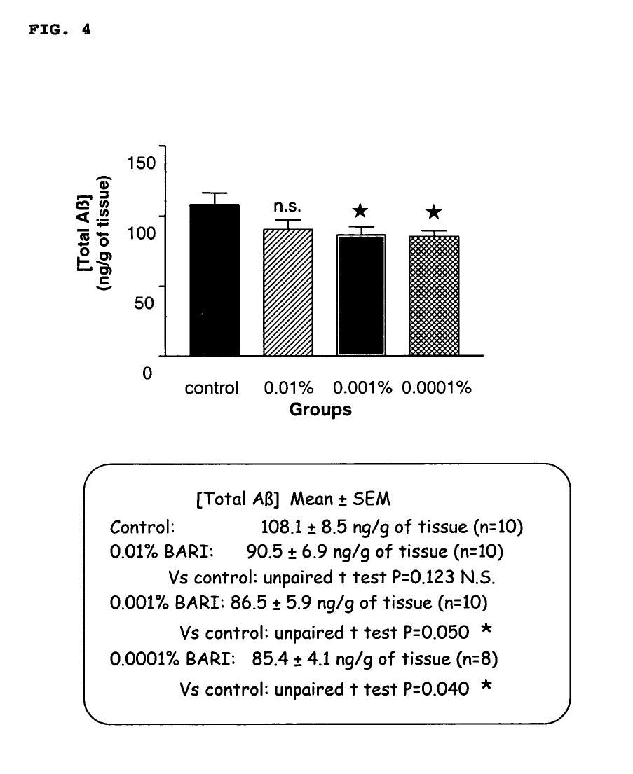

c) Experimental Test No. 3 (FIG. 4)

Under the same experimental conditions, the treatment with various doses of compound A revealed that it was possible to reduce up to at least a factor of 100 the dose of compound A (that is a supplement for the regime with 0.0001%) while retaining the effect of reduction on the cerebral levels of total Aβ peptide. Indeed, the levels of total Aβ were reduced by 21% for 0.0001% of compound A [85.4±4.1 ng/g of tissue (n=8) compared with the control group at 108.1±8.5 ng/g of tissue (n=10), unpaired t test, p=0.04], by 20% for 0.001% of compound A [86.5±5.9 ng/g of tissue (n=10), p=0.050] and by 16% for 0.01% of compound A [90.5±6.9 ng/g of tissue (n=10), p=0.123, ns] (FIG. 4).

1. A pharmaceutical composition for the prevention or treatment of Alzheimer's disease, comprising an effective amount of one or more compounds which are biliary acid reuptake inhibitors (BARI), and one or more pharmaceutically acceptable carriers, diluents or excipients, or a mixture thereof.

2. The composition as set forth in claim 1 wherein the BARI is a compound of formula (IA): 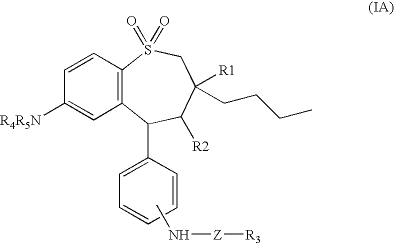
wherein:
R¹ is methyl, ethyl, propyl or butyl;
R² is H, OH, NH₂, or NH—(C₁-C₆)alkyl;
R³ is a saccharide, disaccharide, trisaccharide or quadrisaccharide radical, wherein said radicals are optionally mono- or polysubstituted with a sugar protective group;
R⁴ is methyl, ethyl, propyl or butyl;
R⁵ is methyl, ethyl, propyl or butyl;
Z is —(C═O)ₙ—(C₀-C₁₆)-alkyl; —(C═O)ₙ—(C₀-C₁₆)-alkyl-NH; —(C═O)ₙ—(C₀-C₁₆)-alkyl-O; —(C═O)ₙ—(C₀-C₁₆)-alkyl-(C═O)ₘ—; or a covalent bond;
n is 0 or 1;
m is 0 or 1;
or a pharmaceutically acceptable addition salt thereof, or a physiologically functional derivative thereof.

3. The composition as set forth in claim 2 wherein the compound is having the following formula:

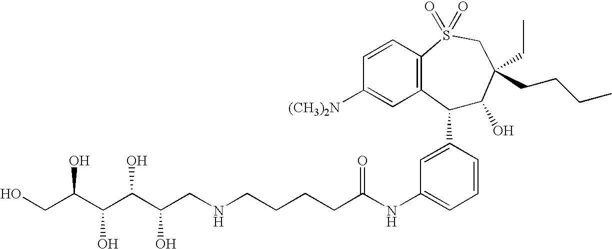

4. The composition as set forth in claim 1 wherein the BARI is a compound of formula (IB): 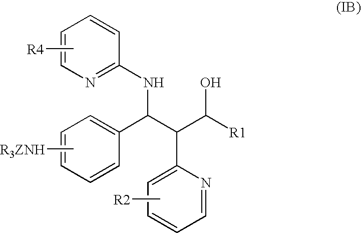
wherein:
R¹ is phenyl or heteroaryl, wherein the aromatic or heteroaromatic ring is unsubstituted or is substituted with one to three independent radicals chosen from F, Cl, Br, I, —OH, —CF₃, —NO₂, —NHR⁹, —NR⁹R¹⁰, —CHO, —CO₂H, —CO₂R¹¹, —COR¹², —(C₁-C₆)-alkyl-OH, —(C₁-C₆)-alkyl-OH-phenyl, —(C₁-C₆)-alkyl-CF₃, —(C₁-C₆)-alkyl-NO₂, —(C₁-C₆)-alkyl-CN, —(C₁-C₆)-alkyl-NH₂, —(C₁-C₆)-alkyl-NHR⁹, —(C₁-C₆)-alkyl-NR⁹R¹⁰, (C₁-C₆)-alkyl-CHO, —(C₁-C₆)-alkyl-CO₂H, —(C₁-C₆)-alkyl-CO₂R¹¹, —(C₁-C₆)-alkyl-COR¹², —O—(C₁-C₆)-alkyl-OH, —O—(C₁-C₆)-alkyl(—OH)-phenyl, —O—(C₁-C₆)-alkyl-CF₃, —O—(C₁-C₆)-alkyl-NO₂, —O—(C₁-C₆)-alkyl-CN, —O—(C₁-C₆)-alkyl-NH₂, —O—(C₁-C₆)-alkyl-NHR⁹, —O—(C₁-C₆)-alkyl-NR⁹R¹⁰, —O—(C₁-C₆)-alkyl-CHO, —O—(C₁-C₆)—N—S₃H, —S₂—CH₃, —O—(C₁-C₆)-alkyl-O—(C₁-C₆)-alkylphenyl, —(C₁-C₆)-alkylthio or pyridyl, wherein one or more hydrogen(s) in the alkyl radicals may be optionally replaced by fluorine, and wherein phenyl or pyridyl may be unsubstituted or monosubstituted by methyl, methoxy or halogen;
R² is H, OH, —CH₂OH, —OMe, —CHO or —NH₂;
R³ is a saccharide, disaccharide, trisaccharide or quadrisaccharide radical, wherein said radical may be unsubstituted or mono- or polysubstituted with a sugar protecting group, HO—SO₂— or (HO)₂—PO—;
R⁴ is H, methyl, F or —OMe;
R⁹, R¹⁰, R¹¹, and R¹² are the same or different, and independently of each other are H or —(C₁-C₈)-alkyl;
Z is a covalent bond or a group chosen from —NH—(C₀-C₁₆)-alkyl-CO—, —O—(C₀-C₁₆)-alkyl-CO—, —(CO)ₘ—(C₀-C₁₆)-alkyl-(CO)ₙ—, an amino acid residue, a diamino acid residue, wherein the amino acid residue or diamino acid residue is unsubstituted, or mono- or polysubstituted by an amino acid-protecting group;
n is 0 or 1;
m is 0 or 1;
or a pharmaceutically acceptable addition salt, or a physiologically functional derivative thereof.

5. The composition as set forth in claim 4 wherein the compound is having the following formula:

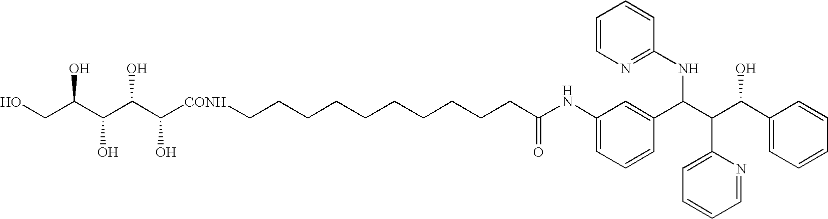

6. The composition as set forth in claim 1 which is administered orally.

7. The composition as set forth in claim 6 which contains from about 0.02 to about 50 mg of the biliary acid reuptake inhibitor.

8. The composition as set forth in claim 1 wherein one or more biliary acid reuptake inhibitors are combined with one or more compounds chosen from HMG-CoA reductase inhibitors, cholesterol uptake inhibitors, cholesterol synthesis inhibitors or γ and β APP secretase inhibitors.

9. The composition as set forth in claim 8 wherein the various active ingredients are administered simultaneously, separately or spaced out over time.

10. A pharmaceutical composition for the prevention or treatment of Alzheimer's disease, comprising an effective amount of one or more compounds which reduce the plasma cholesterol levels without the need to be absorbed in the body after their oral administration, and one or more pharmaceutically acceptable carriers, diluents or excipients, or a mixture thereof.

11. The composition as set forth in claim 10 wherein the compound is of the following formula (IA): 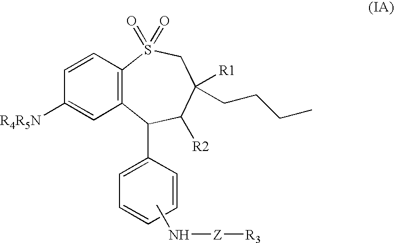
wherein:
R¹ is methyl, ethyl, propyl or butyl;
R² is H, OH, NH₂, or NH—(C₁-C₆)alkyl;
R³ is a saccharide, disaccharide, trisaccharide or quadrisaccharide radical, wherein said radicals are optionally mono- or polysubstituted with a sugar protective group;
R⁴ is methyl, ethyl, propyl or butyl;
R⁵ is methyl, ethyl, propyl or butyl;
Z is —(C═O)ₙ—(C₀-C₁₆)-alkyl; —(C═O)ₙ—(C₀-C₁₆)-alkyl-NH; —(C═O)ₙ—(C₀-C₁₆)-alkyl-O; —(C═O)ₙ—(C₀-C₁₆)-alkyl-(C═O)ₘ—; or a covalent bond;
n is 0 or 1;
m is 0 or 1;
or a pharmaceutically acceptable addition salt thereof, or a physiologically functional derivative thereof.

12. The composition as set forth in claim 11 wherein the compound is having the following formula:

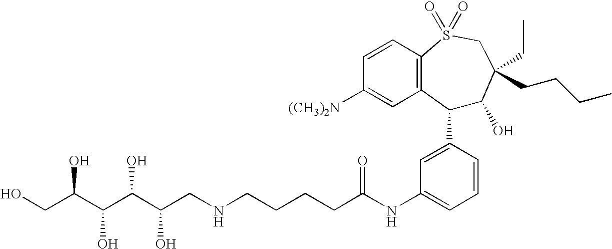

13. The composition as set forth in claim 10 wherein the compound is of the following formula (IB): 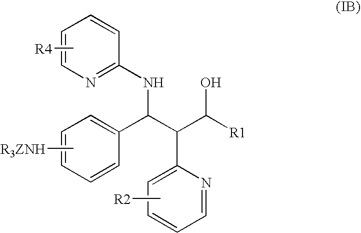
wherein:
R¹ is phenyl or heteroaryl, wherein the aromatic or heteroaromatic ring is unsubstituted or is substituted with one to three independent radicals chosen from F, Cl, Br, I, —OH, —CF₃, —NO₂, —NHR⁹, —NR⁹R¹⁰, —CHO, —CO₂H, —CO₂R¹¹, —COR¹², —(C₁-C₆)-alkyl-OH, —(C₁-C₆)-alkyl-OH-phenyl, —(C₁-C₆)-alkyl-CF₃, —(C₁-C₆)-alkyl-NO₂, —(C₁-C₆)-alkyl-CN, —(C₁-C₆)-alkyl-NH₂, —(C₁-C₆)-alkyl-NHR⁹, —(C₁-C₆)-alkyl-NR⁹R¹⁰, —(C₁-C₆)-alkyl-CHO, —(C₁-C₆)-alkyl-CO₂H, —(C₁-C₆)-alkyl-CO₂R¹¹, —(C₁-C₆)-alkyl-COR², —O—(C₁-C₆)-alkyl-OH, —O—(C₁-C₆)-alkyl(—OH)-phenyl, —(C₁-C₆)-alkyl-CF₃, —O—(C₁-C₆)-alkyl-NO₂, —O—(C₁-C₆)-alkyl-CN, —O—(C₁-C₆)-alkyl-NH₂, —O—(C₁-C₆)-alkyl-NHR⁹, —O—(C₁-C₆)-alkyl-NR⁹R¹⁰, —O—(C₁-C₆)-alkyl-CHO, —O—(C₁-C₆)—N—S₃H, —S₂—CH₃, —O—(C₁-C₆)-alkyl-O—(C₁-C₆)-alkylphenyl, —(C₁-C₆)-alkylthio or pyridyl, wherein one or more hydrogen(s) in the alkyl radicals may be optionally replaced by fluorine, and wherein phenyl or pyridyl may be unsubstituted or monosubstituted by methyl, methoxy or halogen;
R² is H, OH, —CH₂OH, —OMe, —CHO or —NH₂;
R³ is a saccharide, disaccharide, trisaccharide or quadrisaccharide radical, wherein said radical may be unsubstituted or mono- or polysubstituted with a sugar protecting group, HO—SO₂— or (HO)₂—PO—;
R⁴ is H, methyl, F or —OMe;
R⁹, R¹⁰, R¹¹, and R¹² are the same or different, and independently of each other are H or —(C₁-C₈)-alkyl;
Z is a covalent bond or a group chosen from —NH—(C₀-C₁₆)-alkyl-CO—, —O—(C₀-C₁₆)-alkyl-CO—, —(CO)ₘ—(C₀-C₁₆)-alkyl-(CO)ₙ—, an amino acid residue, a diamino acid residue, wherein the amino acid residue or diamino acid residue is unsubstituted, or mono- or polysubstituted by an amino acid-protecting group;
n is 0 or 1;
m is 0 or 1;
or a pharmaceutically acceptable addition salt, or a physiologically functional derivative thereof.

14. The composition as set forth in claim 13 wherein the compound is having the following formula:

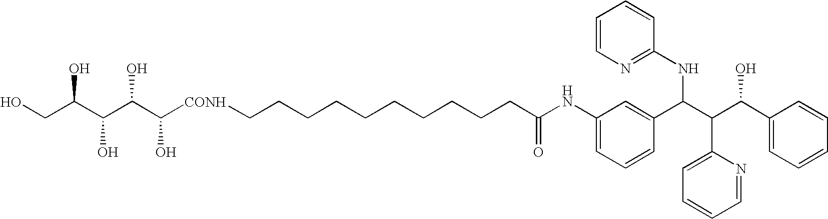

15. The composition as set forth in claim 10 which is administered orally.

16. The composition as set forth in claim 15 which contains from about 0.02 to about 50 mg of the compound.

17. The composition as set forth in claim 10 wherein one or more biliary acid reuptake inhibitors are combined with one or more compounds chosen from HMG-CoA reductase inhibitors, cholesterol uptake inhibitors, cholesterol synthesis inhibitors or γ and β APP secretase inhibitors.

18. The composition as set forth in claim 17 wherein the various active ingredients are administered simultaneously, separately or spaced out over time.

19. A method for the prevention or treatment of Alzheimer's disease in a patient at risk of developing said disease or in the course of developing said disease, comprising administering to said patient an effective amount of a compound having a hypocholesterolemic activity wherein said compound does not penetrate into the body after its oral administration.

20. The method as set forth in claim 19 wherein the compound having a hypocholesterolemic activity and not penetrating into the body is a biliary acid reuptake inhibitor.

21. The method as set forth in claim 20, wherein the biliary acid reuptake inhibitor is a compound of formula (IA): 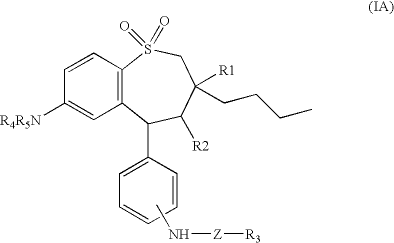
wherein:
R¹ is methyl, ethyl, propyl or butyl;
R² is H, OH, NH₂, or NH—(C₁-C₆)alkyl;
R³ is a saccharide, disaccharide, trisaccharide or quadrisaccharide radical, wherein said radicals are optionally mono- or polysubstituted with a sugar protective group;
R⁴ is methyl, ethyl, propyl or butyl;
R⁵ is methyl, ethyl, propyl or butyl;
Z is —(C═O)ₙ—(C₀-C₁₆)-alkyl; —(C═O)ₙ—(C₀-C₁₆)-alkyl-NH; —(C═O)ₙ—(C₀-C₁₆)-alkyl-O; —(C═O)ₙ—(C₀-C₁₆)-alkyl-(C═O)ₙ—; or a covalent bond;
n is 0 or 1;
m is 0 or 1;
or a pharmaceutically acceptable addition salt thereof, or a physiologically functional derivative thereof.

22. The method as set forth in claim 21 wherein the compound is having the following formula:

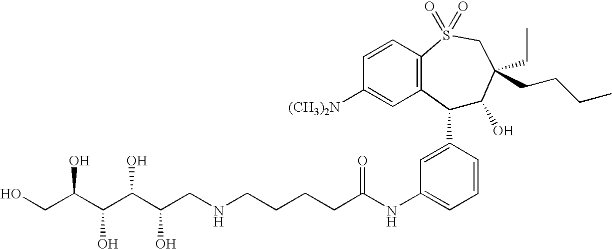

23. The method as set forth in claim 20 wherein the biliary acid reuptake inhibitor is a compound of formula (IB): 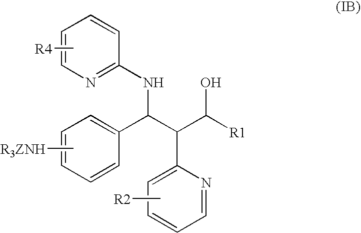
wherein:
R¹ is phenyl or heteroaryl, wherein the aromatic or heteroaromatic ring is unsubstituted or is substituted with one to three independent radicals chosen from F, Cl, Br, I, —OH, —CF₃, —NO₂, —NHR⁹, —NR⁹R¹⁰, —CHO, —CO₂H, —CO₂R¹¹, —COR¹², —(C₁-C₆)-alkyl-OH, —(C₁-C₆)-alkyl-OH-phenyl, —(C₁-C₆)-alkyl-CF₃, —(C₁-C₆)-alkyl-NO₂, —(C₁-C₆)-alkyl-CN, —(C₁-C₆)-alkyl-NH₂, —(C₁-C₆)-alkyl-NHR⁹, —(C₁-C₆)-alkyl-NR⁹R¹⁰, —(C₁-C₆)-alkyl-CHO, —(C₁-C₆)-alkyl-CO₂H, —(C₁-C₆)-alkyl-CO₂R¹¹, —(C₁-C₆)-alkyl-COR¹², —O—(C₁-C₆)-alkyl-OH, —O—(C₁-C₆)-alkyl(—OH)-phenyl, —O—(C₁-C₆)-alkyl-CF₃, —O—(C₁-C₆)-alkyl-NO₂, —O—(C₁-C₆)-alkyl-CN, —O—(C₁-C₆)-alkyl-NH₂, —O—(C₁-C₆)-alkyl-NHR⁹, —O—(C₁-C₆)-alkyl-NR⁹R¹⁰, —O—(C₁-C₆)-alkyl-CHO, —O—(C₁-C₆)—N—S₃H, —S₂—CH₃, —O—(C₁-C₆)-alkyl-O—(C₁-C₆)-alkylphenyl, —(C₁-C₆)-alkylthio or pyridyl, wherein one or more hydrogen(s) in the alkyl radicals may be optionally replaced by fluorine, and wherein phenyl or pyridyl may be unsubstituted or monosubstituted by methyl, methoxy or halogen;
R² is H, OH, —CH₂OH, —OMe, —CHO or —NH₂;
R³ is a saccharide, disaccharide, trisaccharide or quadrisaccharide radical, wherein said radical may be unsubstituted or mono- or polysubstituted with a sugar protecting group, HO—SO₂— or (HO)₂—PO—;
R⁴ is H, methyl, F or —OMe;
R⁹, R¹⁰, R¹¹, and R¹² are the same or different, and independently of each other are H or —(C₁-C₈)-alkyl;
Z is a covalent bond or a group chosen from —NH—(C₀-C₁₆)-alkyl-CO—, —O—(C₀-C₁₆)-alkyl-CO—, —(CO)ₘ—(C₀-C₁₆)-alkyl-(CO)ₙ—, an amino acid residue, a diamino acid residue, wherein the amino acid residue or diamino acid residue is unsubstituted, or mono- or polysubstituted by an amino acid-protecting group;
n is 0 or 1;
m is 0 or 1;
or a pharmaceutically acceptable addition salt, or a physiologically functional derivative thereof.

24. The method as set forth in claim 23 wherein the compound is having the following formula:

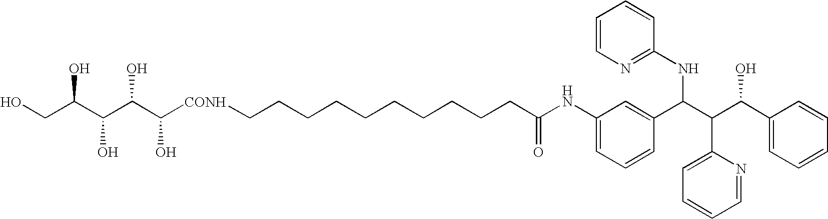

25. The method as set forth in claim 19 wherein the compound is administered orally.

26. The method as set forth in claim 25 wherein the compound is administered in an amount from about 0.02 mg to about 50 mg.

27. The method as set forth in claim 20 wherein one or more biliary acid reuptake inhibitors are administered in combination with one or more compounds chosen from HMG-CoA reductase inhibitors, cholesterol uptake inhibitors, cholesterol synthesis inhibitors or γ and β APP secretase inhibitors.

28. The composition as set forth in claim 27 wherein the various active ingredients are administered simultaneously, separately or spaced out over time.
